(19) United States
(12) Patent Application Publication
(10) Pub. No.: US 2024/0400984 A1
(43) Pub. Date: Dec. 5, 2024

(54) Treatment of Neuronal Absence Disease by Transdifferentiating Treatment

(71) Applicant: Shanghai GeneMagic BioSciences Co., Ltd., Shanghai (CN)

(72) Inventors: Haibo Zhou, Shanghai (CN); Canbin Feng, Shanghai (CN); Zhengzheng Xu, Shanghai (CN); Ni Gao, Shanghai (CN); Xinde Hu, Shanghai (CN)

(21) Appl. No.: 18/182,664

(22) Filed: Mar. 13, 2023

(30) Foreign Application Priority Data
Mar. 14, 2022 (CN) 202210248958.7

(51) Int. Cl. C12N 5/0793; C12N 9/22

(52) U.S. Cl. CPC C12N 5/0619; C12N 9/22; C12N 2310/20; C12N 2501/60; C12N 2506/00

(57) ABSTRACT

This invention provides the use of one or more inhibitors reducing the expression or activity of the genes, the genes's RNA, or the genes's encoding proteins to treat the diseases associated with neuronal functional loss or the neuronal death. The genes are selected from: Plpp7, Fam126a, Gprc5c, Tmed4, Tle6, Psmd5, Mastl, Ssr3, Rhoa, Rfx8, Rbm10, Hnrupa3, Prpf6, Pou3f3, Ncoal, Ccdc8, Adck1, Gjb2, Smad9, Nr2e1, Atp10b, Nid1, Tmcc3, Rad21, Amigo1, Cep192, Sepp1, Klf12, Nxf1, Trp53inp2, Phlpp1, Ptpdc1, Pebp1, Gm22174, Gm26117, Mir873a, Mir1900, Gm22414, Khdc4, Hnrnpa0, Hnrnph2, Srrm1, Hnrnpf, Srsf4, Mbnl1, Zbtb42, Kcmf1, Gtf2i, Chgb, Fos, Kat2a, Tsg101, Hmgb4, Junb, Cdx2, Cers2, Rhox6, Thap3, Zscan25.

Treatment of neuronal absence disease by transdifferentiating treatment

### RELATED APPLICATION

This application claims the benefit of CN application Ser. No. 20/221,0248958.7 and filed Mar. 14, 2022, which is hereby incorporated by reference in its entirety.

FIELD

This invention belongs to the field of translational medicine. Specifically, this invention involves the technology of transforming the non-nervous cells into neurons or neural precursor cells through reducing the expression or activity of the cell transformation factors, and the use of the inhibitors reducing the expression or activity of the cell transformation factors to treat the diseases associated with neuronal function loss or neuronal death.

### BACKGROUND OF THE INVENTION

Cell trans-differentiation refers to the process where a type of differentiated cell transforms into another type of differentiated cell having different structure and function through gene-selective expression or gene reprogramming.

Recent studies have shown that certain neuron-specific trans-differentiation factors can convert non-neuronal cells (such as fibroblasts) into neurons. Converting non-neuronal cells into neurons is expected to be useful for diseases associated with neuronal functional loss or death, such as Parkinson's disease, stroke, visual system diseases related to RGC or photoreceptor cell dysfunction or death, dementia, blindness, deafness, Huntington's chorea, schizophrenia, depression, sleep disorders, traumatic brain injury; etc.

Currently, there are two main regeneration technologies used to treat such neurological diseases. One is the cell transplantation, such as using stem cell transplantation, or using the neural precursor cells differentiated from autologous iPSCs to transplante to treat neurological system diseases such as Parkinson's or stroke. However, these treatments are expensive, require personalized treatment, have high processing costs, and are difficult to commercialize. The other one is in situ trans- differentiation. For example, in the nervous system, visual system or auditory system, the scientists are trying to convert glial cells into functional neurons to treat neurodegenerative diseases or functional neuronal loss diseases, such as stroke, ALS, deafness, blindness, etc. However, the mammalian's terminal cells, such as glial cells, are difficult to dedifferentiate or transdifferentiate into different types of cells, especially in situ trans-differentiation in vivo. Currently, there are few methods that can efficiently convert non-neuronal cells, especially glial cells, into neurons. Moreover, it is even more challenging to transdifferentiate the non-neuronal cells into specific types neurons directly. Therefore, how to directly transdifferentiate non-neuronal cells into neurons is still an urgent problem that needs to be solved in the field of neural regeneration.

### SUMMARY OF THE INVENTION

The purpose of the present invention is to provide a method for converting non-neuronal cells into neurons, which can transdifferentiate non-neuronal cells into neuronal cells.

For achieving the above purpose, the present invention provides the following technical solutions.

In the first aspect, the present invention provides a method for converting non-neuronal cells into neurons or neural precursor cells. The method includes reducing the expression or activity of cell trans-differentiation factors, wherein the cell trans-differentiation factor are at least one gene, or at least one gene's RNA, or at least one gene-encoded protein selected from Plpp7, Fam126a, Gprc5c, Tmed4, Tlc6, Psmd5, Mastl, Ssr3, Rhoa, Rfx8, Rbm10, Hnrnpa3, Prpf6, Pou3f3, Ncoal, Ccdc8, Adck1, Gjb2, Smad9, Nr2cl, Atp10b, Nidl, Tmcc3, Rad21, Amigo1, Cep192, Sepp1, Klf12, Nxf1, Trp53inp2, Phlppl, Ptpdcl, Pebpl, Gm22174, Gm26117, Mir873a, Mir1900, Gm22414, Khdc4, Hnrnpa0, Hnrnph2, Srrm1, Hnrnpf, Srsf4, Mbnl1, Zbtb42, Kcmfl, Gtf2i, Chgb, Fos, Kat2a, Tsg 101, Hmgb4, Junb, Cdx2, Cers2, Rhox6, Thap3, or Zscan25. Preferably, the non-neuronal cells are selected from stem cells, progenitor cells, or terminally differentiated cells. More preferably, the non-neuronal cells are mammalian non-neuronal cells, such as human, non-human primate, mouse, or rat non-neuronal cells.

In some preferred embodiments, the stem cells mentioned in this aspect are embryonic stem cells, neural stem cells, or induced pluripotent stem cells.

In some preferred embodiments of any of the above methods, the terminally differentiated cells mentioned in this aspect are glial cells. Preferably, the glial cells are selected from astrocytes, oligodendrocytes, microglia, NG2 cells, Müller glia cells, glioblastoma cells, or spiral ganglion glia cells. More preferably, the glial cells are selected from astrocytes, Müller glia cells, or spiral ganglion glia cells.

In some preferred embodiments of any of the above methods, the above-mentioned methods can be to culture non-neuronal cells in vitro, convert non-neuronal cells into neurons or neuronal precursor cells in vitro by reducing the expression or activity of cell trans-differentiation factors: or to induce non-neuronal cells in vivo to convert into neurons or neuronal precursor cells in vivo by reducing the expression or activity of cell trans-differentiation factors.

In some preferred embodiments of any of the above methods, the non-neuronal cells mentioned above are glial cells that are converted into neurons or neuronal precursor cells in vivo.

In some preferred embodiments of any of the above methods, the non-neuronal cells mentioned above are astrocytes that are converted into dopamine neurons or dopamine precursor cells in vivo and/or in vitro.

In some preferred embodiments of any of the above methods, the non-neuronal cells mentioned above are Müller glia cells that are converted into dopamine neurons or dopamine precursor cells in vivo and/or in vitro.

In some preferred implementations of any of the above methods, the non-neuronal cells referred to in any of the above methods are Müller glial cells, and the neuronal cells are RGCs (retinal ganglion cells) or photoreceptor cells.

In some preferred implementations of any of the above methods, the non-neuronal cells referred to in the above methods are spiral ganglion glial cells, and the neuronal cells are cochlear nerve cells.

In some preferred implementations of any of the above methods, the method of reducing the expression or activity of cell trans-differentiation factors includes administering an inhibitory substance that reduces the expression or activity of cell trans-differentiation factors, wherein the inhibitory substance includes at least one gene or RNA or protein inhibitor that reduces the expression or activity of Plpp7, Fam 126a, Gprc5c, Tmed4, Tlc6, Psmd5, Mastl, Ssr3, Rhoa, Rfx8, Rbm10, Hnrnpa3, Prpf6, Pou3f3, Ncoal, Ccdc8, Adckl, Gjb2, Smad9, Nr2cl, Atp10b, Nidl, Tmcc3, Rad21, Amigol, Cep192, Seppl, Klf12, Nxfl, Trp53inp2, Phlppl, Ptpdcl, Pebpl, Gm22174, Gm26117, Mir873a, Mir1900, Gm22414, Khdc4, Hnrnpa0, Hnrnph2, Srrm1, Hnrnpf, Srsf4, Mbnl1, Zbtb42, Kcmf1, Gtf2i, Chgb, Fos, Kat2a, Tsg101, Hmgb4, Junb, Cdx2, Cers2, Rhox6, Thap3, or Zscan25. Preferably, the inhibitor is selected from gene editing tools that regulate the expression of cell trans-differentiation factors, epigenetic regulation tools, antibodies, small molecules, mRNA, microRNA, siRNA, shRNA, antisense oligonucleotides, binding proteins or protein domains, peptides, nucleic acid aptamers, PROTACs, expression vectors containing promoters, protein mimetics, and artificially synthesized or modified inhibitors above or combinations thereof. More preferably, the gene editing tools include:

(a) a gene editing system or its expression vector, wherein the gene editing system is selected from: CRISPR system (including CRISPR/dCas system), ZFN system, TALEN system, RNA editing system, or a combination thereof: and/or (b) one or more required gRNA or its expression vector, wherein the gRNA guides the gene editing protein to specifically bind to the DNA or RNA of the target genc.

In some preferred embodiments of the above methods, the method of reducing the expression or activity of cell trans-differentiation factors uses CRISPR system to reduce the expression or activity of the cell trans-differentiation factors. Preferably, the CRISPR gene editing tool includes nucleic acid encoding Cas enzyme or Cas enzyme functional domain and gRNA targeting the cell trans-differentiation factors. More preferably; the Cas enzyme is Cas13d, CasRx, Cas13X, Cas13a, Cas13b, Cas13c, or Cas13Y: more preferably; the Cas enzyme is CasRx, Cas13X, or Cas13Y: and most preferably; the Cas enzyme is CasRx.

In some preferred embodiments, the inhibitory substance described in any of the aforementioned methods further comprises a carrier. Preferably, the carrier is a viral vector, lipid nanoparticle (LNP), liposome, cationic polymer (such as PEI), nanoparticle, exosome, or virus-like particle: more preferably, the carrier is an AAV vector or lipid nanoparticle.

In some preferred embodiments, the astrocyte described in any of the aforementioned methods are derived from the brain or spinal cord. Preferably, the brain is selected from the cerebrum, midbrain, cerebellum, or brainstem: more preferably from the striatum or substantia nigra.

In some preferred embodiments, the Müller glial cells described in any of the aforementioned methods are derived from the retina.

In some preferred embodiments, the spiral ganglion glial cells described in any of the aforementioned methods are derived from the inner car or vestibule.

In some preferred embodiments, the neuronal cells are mammalian neurons, such as human, non-human primate, rat, or mouse neurons. The neuronal cells are preferably dopamine neurons, 5- HT neurons, NE neurons, ChAT neurons, GABA neurons, glutamatergic neurons, motor neurons, photoreceptor cells (such as rod cells and cone cells), retinal ganglion cells (RGCs), cochlear nerve cells (such as spiral ganglion neurons and vestibular neurons), or medium spiny neurons (MSNs), or combinations thereof. More preferably, the neuronal cells are dopamine neurons, RGCs, or photoreceptor cells. In a further preferred embodiment, the non-neuronal cells are astrocyte and the neuronal cells are dopamine neurons: or the glial cells are Müller glial cells and the neuronal cells are RGCs or photoreceptor cells.

In some preferred embodiments, the cell trans-differentiation factors mentioned in any of the previous methods are selected from at least one gene, RNA, or protein encoded by at least one of Plpp7, Fam126a, Gprc5c, Tmed4, Tlc6, Psmd5, Mastl, Ccdc8, Adck1, Gjb2, Smad9, Nr2cl, Atp10b, Nid1, Tmcc3, Rad21, Amigol, Rbm10, and Hnrnpa3: preferably; the cell trans-differentiation factors are selected from at least one gene, RNA, or protein encoded by at least one of Amigol, Fam126a, Gjb2, or Gprc5c.

In the second aspect of the present invention, an inhibitor for reducing the expression or activity of the cell trans-differentiation factors are provided to prepare the drug treating the diseases related to neuronal function loss (also said neuronal dysfunction) or neuronal death. The cell trans- differentiation factors selected from at least one gene, or the gene's RNA, or the gene's protein of Plpp7. Fam126a, Gprc5c. Tmed4, Tlc6, Psmd5, Mastl, Ssr3, Rhoa, Rfx8, Rbm10, Hnrnpa3, Prpf6. Pou3f3, Ncoal, Ccdc8, Adckl, Gjb2, Smad9, Nr2cl, Atp10b, Nidl, Tmcc3, Rad21, Amigol, Cep192, Scpp1, Klf12, Nxfl, Trp53inp2, Phlpp1, Ptpdcl, Pebpl, Gm22174, Gm26117, Mir873a, Mir1900, Gm22414, Khdc4, Hnrnpa0), Hnrnph2, Srrm1, Hnrnpf, Srsf4, Mbnll, Zbtb42, Kcmfl, Gtf2i, Chgb, Fos, Kat2a, Tsg101, Hmgb4, Junb, Cdx2, Cers2, Rhox6, Thap3, Zscan25. Preferably, the cell trans-differentiation factors are selected from at least one gene, RNA, or protein encoding gene of Plpp7, Fam 126a, Gprc5c, Tmed4, Tle6, Psmd5, Mastl, Ccdc8, Adck1, Gjb2, Smad9, Nr2el, Atp10b, Nidl, Tmcc3, Rad21, Amigol, Rbm10, or Hnrnpa3. More preferably, the cell trans- differentiation factors are selected from at least one gene, RNA, or protein encoding gene of Amigo1. Fam126a, Gjb2, or Gprc5c.

In some preferred embodiments, the drug for the aforementioned use is formulated into a formulationfor in vivo administration to the nervous system, visual system, and auditory system, such as administered in vivo to the striatum, substantia nigra, ventral tegmental area, spinal cord. hypothalamus, dorsal midbrain, cerebral cortex, hippocampus, cerebellum, subretinal space, vitreous cavity, inner car cochlea, and vestibule, preferably to the striatum, substantia nigra, subretinal space, and vitreous cavity.

In some preferred embodiments, the discases mentioned above are referred to as neurological diseases. The preferred neurological diseases include Parkinson's disease, visual system diseases related to RGC or photoreceptor dysfunction or death, stroke, Alzheimer's disease, brain injury; Huntington's disease, epilepsy, depression, sleep disorders, cerebral ischemia, motor neuron discase, amyotrophic lateral sclerosis, spinal muscular atrophy, ataxia. PloyQ disease, schizophrenia. addiction, Pick's disease, blindness, and deafness. Parkinson's disease and visual system diseases related to RGC or photoreceptor dysfunction or death are even more preferred. The visual system discases related to RGC dysfunction or death include visual impairment caused by RGC cell death. glaucoma. age-related RGC lesions. optic nerve injury: age-related macular degeneration (AMD). diabetes-related retinal lesions. retinal ischemia or bleeding. Leber's hereditary optic neuropathy. or a combination thereof. The visual system diseases related to photoreceptor dysfunction or death include photoreceptor degeneration or death caused by injury or degenerative discases. macular degeneration. retinal pigment degeneration. blindness related to diabetes. night blindness. color blindness, hereditary blindness. congenital achromatopsia. or a combination thereof.

In some preferred embodiments. the neurons mentioned in any of the above uses include dopamine neurons. 5-HT neurons. NE neurons. ChAT neurons. GABA neurons. glutamatergic neurons. motor neurons. photoreceptors (such as rods and cones). retinal ganglion cells (RGCs). cochlear nerve cells (such as spiral ganglion cells and vestibular neurons). or medium spiny neurons (MSNs). or combinations thereof. Dopamine neurons. RGCs. and photoreceptors are preferred.

In some preferred embodiments. the inhibitor mentioned in any of the above uses contacts non- neuronal cells in vitro, causing them to convert into neurons or neuronal precursor cells: or the inhibitor is directly administered to the subject in need to induce non-neuronal cells in vivo to convert into neurons or neuronal precursor cells.

In some preferred embodiments. the aforementioned inhibitors for any of the purposes described above are selected from: gene editors. epigenetic regulators. antibodies. small molecules. mRNA. microRNA. siRNA. shRNA. antisense oligonucleotides. binding proteins or protein domains. peptides. nucleic acid aptamers. PROTACs. expression vectors containing promoters. protein mimetics. or artificially synthesized or modified combinations thereof. Preferably, the genc editing tools include: (a) a gene editing system or its expression vector. where the gene editing system is selected from: the CRISPR system (including the CRISPR/dCas system). the ZFN system. the TALEN system. the RNA editing system, or their combinations: and/or (b) one or more required gRNAs or their expression vectors, where the gRNA guides the gene editing protein to specifically bind to the DNA or RNA of the target gene.

In some more preferred embodiments. the CRISPR system for the aforementioned purposes contains nucleic acids encoding the cas enzyme or functional domain of the cas enzyme and gRNA targeting the cell trans-differentiation factors: more preferably. the cas enzyme is Cas 13d. CasRx. Cas13X. Cas13a. Cas13b. Cas13c. or Cas13Y: even more preferably. the cas enzyme is CasRx. Cas13X. or Cas13Y: and most preferably. the cas enzyme is CasRx.

In the third aspect of the present invention, a pharmaceutical composition or a kit is provided. which comprises an inhibitor as described in any of the uses in the second aspect of the present invention. Preferably. the pharmaceutical composition or kit further comprises an expression vector. More preferably. the expression vector is a viral vector. a lipid nanoparticle (LNP). a liposome. a cationic polymer (such as PEI). a nanoparticle. an extracellular vesicle. or a virus-like particle. Even more preferably. the expression vector is a viral vector or a lipid nanoparticle. Furthermore, even more preferably. the viral vector is an adeno-associated virus (AAV) vector. a self-complementary adeno-associated virus vector (scAAV), an adenovirus vector, a lentivirus vector, a retrovirus vector. a herpesvirus vector. an SV40 vector, a vaccinia virus vector, or a combination of at least two of them, and even more preferably. the viral vector is an AAV vector.

In some preferred embodiments. the inhibitor in the aforementioned pharmaceutical composition or kit comprises: (a) a gene editing system or its expression vector, wherein the editing system includes: CRISPR system (including CRISPR/dCas system). ZFN system. TALEN system. RNA editing system. or a combination thereof: and/or (b) one or more gRNAs or their expression vectors, wherein the gRNA guides gene editing proteins to specifically bind to the DNA or RNA of the gene. and preferably. the CRISPR gene editing system (including DNA and RNA-targeting CRISPR system) is preferred. Preferably. the pharmaceutical composition or kit comprises only a single type of gRNA that targets the DNA or mRNA sequence or 2. 3. 4. 5. 6. 7. 8. 9. 10. 11. or 12 different types of gRNA that target the DNA or mRNA sequence, or the gRNA expression vector encodes only a single type of gRNA that targets the mRNA sequence or 2. 3. 4. 5. 6. 7. 8. 9. 10. 11. or 12 different types of gRNA that target the mRNA sequence.

In some preferred embodiments . . . the inhibitor in the aforementioned drug combination or kit is selected from the gene editing tool of CRISPR. including the nucleic acid encoding the Cas protein. the promoter of the Cas protein. the gRNA targeting the cell trans-differentiation factors. and the promoter of the gRNA. Preferably, the CRISPR gene editing tool includes:

i) a nucleic acid sequence encoding the gene editing protein. which is operably linked to the promoter that induces the expression of the gene editing protein. The promoter is a broad-spectrum promoter or a specific promoter. The broad-spectrum promoter is selected from CMV. CBH. CAG. PGK. SV40. EFIA. EFS, and pGlobin promoters. The specific promoter is preferably a glial cell- specific promoter or a Muller glial cell (MG) cell-specific promoter. More preferably. the glial cell- specific promoter is selected from GFAP promoter. ALDHILI promoter. EAATI/GLAST promoter. glutamine synthetase promoter. S100ß promoter. EAAT2/GLT-1 promoter. NG2 promoter. CD68 promoter. F4/80 promoter. or the MG cell-specific promoter is selected from GFAP promoter. ALDHILI promoter. Glast (also known as Slcla3) promoter. and Rlbpl promoter: and ii) at least one nucleic acid sequence encoding gRNA targeting the mRNA or DNA sequence. the nucleic acid sequence is operably linked to the promoter that induces the expression of gRNA in mammalian cells, such as the U6 promoter.

In some preferred implementations, the aforementioned combination of drugs or kit is locally administered in the body of the subject in need. Preferably, the neurological system is selected from any of the following locations: retina. striatum. substantia nigra. inner car. spinal cord. prefrontal cortex. motor cortex. thalamus, ventral tegmental area (VTA). hippocampus. cerebellum. brainstem, or inner car cochlea or vestibule. More preferably. the drug is administered to the striatum, substantia nigra. retina. and vitreous cavity of the subject in need. Alternatively. the combination of drugs or kit induces glial cells to transform into neuron cells in vitro and then gives the neuron cells to the individual in need. Preferably, the glial cells are selected from astrocytes. oligodendrocytes. microglia. NG2 cells. Muller glia cells. glioma cells. or spiral ganglion glia cells. More preferably. the glial cells are selected from astrocytes. Muller glia cells. or spiral ganglion glia cells.

In some preferred implementations, the aforementioned combination of drugs or kit further includes i) one or more dopamine neuron-related factors. or ii) at least one expression vector for expressing one or more dopamine neuron-related factors in the glial cells. Preferably. the dopamine neuron-related factors are selected from a combination of at least one of the following: Lmxla. Lmxlb. FoxA2. Nurr1. Pitx3. Gata2. Gata3. FGF8. BMP. En1. En2. PET1. Pax family proteins.

SHH, Wnt family proteins, and TGF-ß family proteins.

In some preferred implementations, any of the aforementioned drug combinations or kit, the composition further includes i) one or more factors selected from B-catenin, Oct4, Sox2, Klf4, Crx, Brn3a, Brn3b, Math5, Nr2e3, and Nrl, and/or ii) at least one expression vector for expressing one or more factors selected from B-catenin, Oct4, Sox2, Klf4, Crx, Brn3a, Brn3b, Math5, Nr2e3, and Nrl in glial cells.

In some preferred implementations, the aforementioned inhibitor is formulated for cell transfection, cell infection, endocytosis, injection, intracranial administration, spinal cord administration, intraocular administration, intra-aural administration, inhalation, extraintestinal administration, intravenous administration, intramuscular administration, subcutaneous administration, topical administration or oral administration, as well as for inducing differentiation in vitro, trans-differentiation or reprogramming in vitro, and then transplanting differentiated, transdifferentiated or reprogrammed cells back into the body.

In some preferred implementations, any of the aforementioned drug combinations or kit, a) the differentiation efficiency of stem cells, iPSCs, progenitor cells or germ cells is at least 0.1%, 1%, 10%, 20%, 30%, 40%, 50%, 60% or higher: b) the trans-differentiation efficiency of glial cells is at least 0.1%, 1%, 10%, 20%, 30%, 40%, 50%, 60% or higher.

The fourth aspect of the present invention provides a method for screening multiple factors, such as cell trans-differentiation factors, comprising the following steps:

(1) Construction of a screening cell line, such as the CAG-LSL-CasRx screening cell line:

(2) Knock-in of a reporter system at the Tubb3 locus, such as an EGFP fluorescence reporter system, where the Tubb3 promoter activates the reporter system, such as EGFP expression, upon differentiation of the cells into neurons:

(3) Construction of a target lentiviral library; Lenti-gRNAs-Cre, where gRNAs designed against selected genes are constructed into the Lenti-Cre backbone vector:

(4) Infection of the screening cell line with the Lenti-gRNAs-Cre library, followed by induction of differentiation in differentiation induction medium, and enrichment of factors that promote the differentiation of the cell line into neurons by flow cytometry: and (5) Construction of each enriched factor into a lentiviral vector, followed by infection of the screening cell line with the lentivirus and induction of differentiation.

In the fifth aspect of the present invention, a screening cell line is provided, which contains a controllable expression CasRx clement. CasRx is not expressed in the absence of Cre, but is expressed when LSL is excised in the presence of Cre. Preferably, the screening cell line is a CAG- LSL-CasRx screening cell line, which has been knocked into the Rosa26 locus through homologous recombination of the CAG-LSL-CasRx-PloyA core element. More preferably, the cell line is derived from stem cells, IPS cells, progenitor cells, glial cells, fibroblasts, or other cells, and embryonic stem cells are more preferred.

In some preferred embodiments, the method of screening multiple factors provided in the fourth aspect of the present invention, or the screening cell line described in the fifth aspect of the present invention, is used to screen for factors promoting neural differentiation, neural transdifferentiation, pancreatic cell differentiation, cardiac cell differentiation, blood cell differentiation, chondrocyte differentiation, immune cell differentiation, adipocyte differentiation, etc., and neural differentiation factors and neural transdifferentiation factors are preferred.

It should be understood that, within the scope of the present invention, the various technical features described above and the specific technical features described in the following embodiments can be combined with each other to form new or preferred technical solutions. Due to space limitations, they are not listed one by one here.

### BRIEF DESCRIPTION OF THE DRAWINGS

FIG. 1. Screening of the cell trans-differentiation factors for inducing mESCs to differentiate into neurons. (A) Construction of the CAG-LSL-CasRx screening cell line. The CAG-LSL-CasRx- PolyA core element was knocked into mESCs at the Rosa26 site by homologous recombination. CasRx is not expressed in the absence of Cre, and CasRx is expressed when LSL is excised by Cre enzyme. LSL is loxp-3xSV40 ployA-Loxp. (B) Flowchart of library construction and induction of mESCs cell trans-differentiation factor screening. TUBB3-GFP-CasRx-mESCs represent a stable mESCs cell line with both the TUBB3-EGFP reporting system and the CasRx system (CAG-LSL- CasRx). After infecting with lentiviral libraries (Lenti-U6-gRNAs-EFla-Cre), differentiation was continued for 12 days, and EGFP-positive cells were sorted by flow cytometry and subjected to high-throughput sequencing analysis. (C) Analysis of gRNA targeting different genes. The enriched gRNAs from high-throughput sequencing were packaged into lentivirus one by one and independently validated, and the proportion of TUBB3-EGFP-positive cells was analyzed by flow cytometry: The higher the proportion of EGFP-positive cells, the more cells differentiated into neurons.

FIG. 2. Verification of the ability of highly enriched gRNAs to induce mESCs to differentiate into neurons. The bright field represents the total number of cells, and the green fluorescence is the TUBB3-EGFP fluorescence signal. The control group did not target any genes, and there was no obvious green fluorescence signal. The gRNA groups targeting Amigol, Fam126a, Gib2, and Gprc5c all had obvious EGFP fluorescence signals. The scale bar is 100 micrometers.

FIG. 3. Verification of the ability of highly enriched gRNAs to induce mESCs to differentiate into mature neurons. The green fluorescence is the TUBB3-EGFP fluorescence signal, the red fluorescence is the MAP2 immunofluorescence signal, and DAPI represents the cell nucleus. The overlay shows the co-localization of the red and green signals. (A) gRNA targeting Gprc5c group: (B) gRNA targeting Amigol group. The scale bar is 100 micrometers.

FIG. 4. The seguence informations of the cell trans-differentiation factors.

### DETAILED DESCRIPTION

The inventor has conducted extensive and in-depth research and unexpectedly discovered that inhibiting the expression or activity of one or more genes, their RNA, or encoded proteins selected from: Plpp7, Fam126a, Gprc5c. Tmed4, Tlc6, Psmd5, Mastl, Ssr3, Rhoa, Rfx8, Rbm10, Hnrnpa3, Prpf6, Pou3f3, Ncoal, Ccdc8, Adck1, Gjb2, Smad9, Nr2cl, Atp10b, Nidl, Tmcc3, Rad21, Amigol, Cep192, Sepp1, Klf12, Nxfl, Trp53inp2, Phlppl. Ptpdcl, Pebpl, Gm22174, Gm26117, Mir873a,

Mir1900, Gm22414, Khdc4, Hnrnpa0, Hnrnph2, Srrm1, Hnmnpf, Srsf4, Mbnl1, Zbtb42, Kcmfl, Gtf2i, Chgb, Fos, Kat2a, Tsg101, Hmgb4, Junb, Cdx2, Cers2, Rhox6, Thap3, Zscan25, can effectively induce non-neuronal cells to differentiate into neuronal cells or neural progenitor cells. Based on this, the present invention has been completed.

Unless otherwise indicated, the definitions of all technology and scientific terms used in this application is the same as that commonly understood by the person skilled in the art. Unless otherwise indicated, conventional methods of chemistry, biochemistry; biophysics, molecular biology; cell biology; genetics, immunology; and pharmacology known to the person skilled in the art are used in the practice of the present invention.

It should be noted that all titles and subtitles used in this application are for convenience only and should not be interpreted as limiting the present invention in any way:

Unless otherwise indicated, the use of exemplary wording such as “such as” in this application is for illustrative purposes only and is not a limitation on the scope of the present invention.

In this application, “one” or “a” or “the” or “this” can represent one or more than one. Unless there are other instructions in this application, the term presented in the form of a “single” order also includes “plural” conditions.

DEFINITION

In this article, when referring to the process of differentiated cells, the terms “trans- differentiation”, “differentiation”, and “reprogramming” can be used interchangeably, referring to the production of specific lineage cells (such as neuronal cells) from different types of non-neuronal cells (such as astrocytes). The methods of the invention are generally defined by a series of steps, which should be understood as stages, in which something is actively happening and/or an action is being performed. Those skilled in the art will understand when steps to be executed and/or performed are simultaneous and/or sequential and/or continuous.

In this article, “stem cells” should be understood as undifferentiated cells with differentiation potential and proliferative capacity (particularly self-renewal capacity). Depending on their differentiation potential, stem cells include subgroups such as pluripotent stem cells (PSCs), multipotent stem cells, unipotent stem cells, embryonic stem cells, etc. In some specific embodiments, stem cells could be embryonic stem cells, neural stem cells, or induced pluripotent stem cells.

In this article, the term “pluripotent stem cells” (PSCs) refers to stem cells that are capable of being cultured in vitro and have the ability to differentiate into any cell lineage belonging to the three germ layers (ectoderm, mesoderm, and endoderm). PSCs can be induced from a fertilized egg, cloned embryos, germ cells, stem cells in tissues, or somatic cells. Examples of PSCs include embryonic stem cells (ESCs), induced pluripotent stem cells (iPSCs or ips), embryonic germ cells (EG cells), and others.

In this article, the term “induced pluripotent stem cells” (iPSCs or ips) can be directly generated from adult cells through a process commonly called reprogramming. By introducing products of a specific set of pluripotency-associated genes, somatic cells can be transformed into PSCs.

In this article, the term “neural precursor cells” refers to a cell that has the potential to develop into a neuron and is in a precursor state of neuronal development.

In this article, the term “non-neuron cells” refers to cells other than neuronal precursor cells and neurons. Preferably, non-neuronal cells are selected from stem cells, ancestral cells, or end- differential cells: more preferably, the non-neuronal cells are selected from stem cells or end- differential cells of the human.

In this article, the term the term “terminally differentiated cell” refers to a cell that has completed its differentiation and generally does not have further differentiation capacity. In some specific embodiments, the end-differential cells are glial cells: preferably, the glial cells are selected from astrocytes, oligodendrocytes, microglia, NG2 cells, Müller glia cells, glioblastoma cells, or spiral ganglion glia cells, more preferably, the glial cells are selected from astrocytes, Müller glia cells, or spiral ganglion glia cells.

In this article, the term “polypeptide” and “protein” are equal and can be used interchangeably. They refer to any amino acid chain and include any modification such as phosphorylation or glycosylation.

In this article, the term “object” refers to any animal (e.g. mammals, birds, reptiles, amphibians. fish), including but not limited to humans, non-human primates, rodents, etc., that become the recipient of specific therapies. Generally, when referring to the object in this article, the terms “object” and “patient” can be used interchangeably.

The term “administration” used in this article refers to various methods of administration such as cell transfection, cell infection, endocytosis, injection, intracranial administration, spinal administration, intraocular administration, intratympanic administration, inhalation, extraintestinal administration, intravenous administration, intramuscular administration, subcutancous administration, topical administration, or oral administration. The pharmaceutical composition of the present invention can be administered alone or in combination with other compounds, excipients, fillers, binders, carriers or other vehicles according to selected routes of administration and standard pharmaceutical practice. Administration can be in the form of a carrier or vehicle, such as injectable solutions including sterile aqueous or non-aqueous solutions or saline solutions, creams, shampoos, capsules, tablets, granules, powders, suspensions, emulsions or microemulsions, patches, liposomes, vesicles, implants, including micro-implants, eye drops, other proteins and peptides, synthetic polymers, microspheres, nanoparticles, viral vectors, lipid nanoparticles (LNPs), cationic polymers (such as PEI), exosomes, etc. In specific embodiments, viral vectors and lipid nanoparticles are preferred. In specific embodiments, the carrier is preferably an AAV vector or lipid nanoparticle.

Cell trans-differentiation factors

In this article, the cell trans-differentiation factor refers to a gene or protein that can influence non-neuronal cells to differentiate into neuronal cells. When the expression or activity of the cell trans-differentiation factors is reduced, it promotes the differentiation of non-neuronal cells into neuronal cells. In some specific embodiments, the cell trans-differentiation factors is selected from Plpp7. Fam 126a, Gprc5c, Tmed4, Tlc6, Psmd5, Mastl, Ssr3, Rhoa, Rfx8, Rbm10, Hnmnpa3, Prpf6, Pou3f3, Ncoal, Ccdc8, Adckl, Gjb2, Smad9, Nr2cl, Atp10b, Nidl, Tmcc3, Rad21, Amigol, Ccp192. Scpp1, Klf12, Nxfl, Trp53inp2, Phlpp1, Ptpdcl, Pebpl, Gm22174, Gm26117, Mir873a,

Mir1900, Gm22414, Khdc4, Hnrnpa0, Hnmnph2, Srrm1, Hnmnpf, Srsf4, Mbnl1, Zbtb42, Kcmfl, Gtf2i, Chgb, Fos, Kat2a, Tsg101, Hmgb4, Junb, Cdx2, Cers2, Rhox6, Thap3, or Zscan25. It may include at least one gene, or at least one RNA of a gene, or at least one protein encoded by a gene.

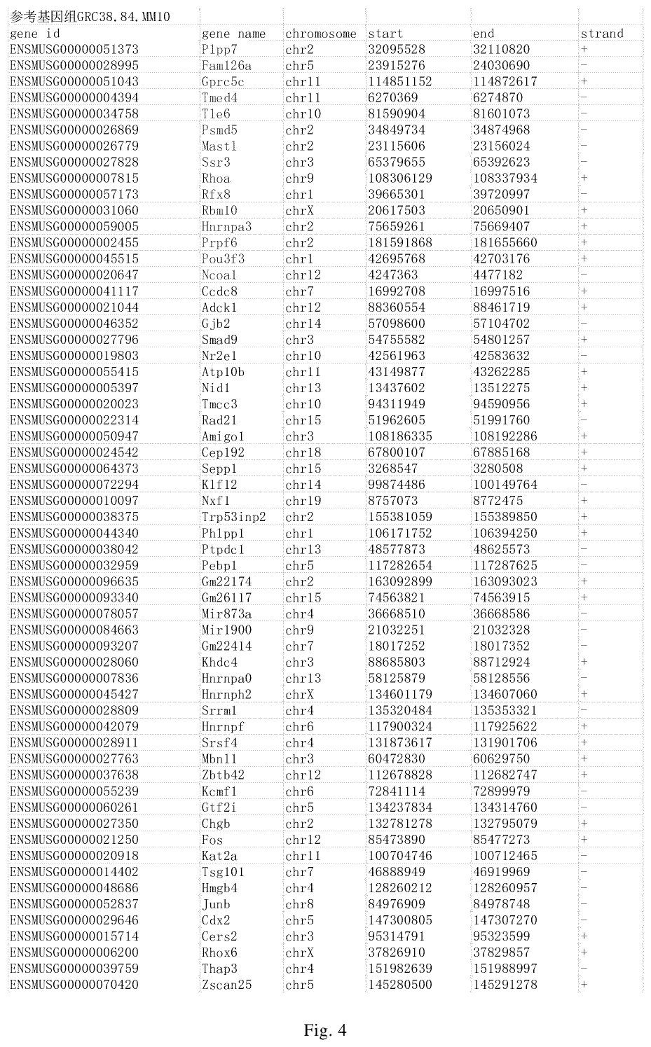

Some informations of the cell trans-differentiation factors show in FIG. 4.

In some specific implementation methods, methods of reducing the expression or activity of cell trans-differentiation factors include administering inhibitors that reduce the expression or activity of cell trans-differentiation factors. The inhibitors can reduce the expression or activity of at least one gene or at least one RNA or at least one protein encoded by a gene, such as Plpp7, Fam 126a, Gprc5c, Tmed4, Tlc6, Psmd5, Mastl, Ssr3, Rhoa, Rfx8, Rbm10, Hnrnpa3, Prpf6, Pou3f3, Ncoal, Ccdc8, Adck1, Gjb2, Smad9, Nr2cl, Atp10b, Nidl, Tmcc3, Rad21, Amigol, Cep 192, Seppl, Klf12, Nxf1, Trp53inp2, Phlppl, Ptpdcl, Pebpl, Gm22174, Gm26117, Mir873a, Mir1900, Gm22414, Khdc4, Hnrnpa0, Hnrnph2, Srrm1, Hnrnpf, Srsf4, Mbnl1, Zbtb42, Kcmfl, Gtf2i, Chgb, Fos, Kat2a, Tsg101, Hmgb4, Junb, Cdx2, Cers2, Rhox6, Thap3, or Zscan25.

In some specific embodiments, the inhibitors may be gene editing tools expressed in the expression of cell transformation factor, nucleic acids expressed in the expression of cell transformation factor, protein that reduces the expression of cell trans-differentiation factors or activity, or active, or active A small molecular compounds that reduce the expression of cell trans- differentiation factors or activity:

In some specific embodiments, the gene editing tools include DNA gene editing and RNA gene editor. In a preferred embodiment, the gene editor of this disclosure includes gene editing protein and optional gRNA.

In some specific embodiments, the gene editing tools are selected from CRISPR gene editing tools, Zinc Finger Nucleases gene editing tools, or TALEN systems.

In some specific embodiments, the nucleic acids regulating the expression of cell trans- differentiation factors are selected from mRNA, microRNA, siRNA, shRNA, antisense oligonucleotide.

In some specific embodiments, the protein reducing the expression or activity of the cell trans- differentiation factor are selected from antibody, polypeptide, PROTAC.

CRISPR Gene Editing Tool

In some specific embodiments, a CRISPR gene editing tool can be used to lower the expression or activity of cell trans-differentiation factors. In some specific embodiments, the CRISPR gene editing tool includes a nucleic acid encoding a Cas enzyme or a functional domain of the Cas enzyme and a guide RNA (gRNA) targeting the cell trans-differentiation factor. More preferably, the Cas enzyme is Cas13d, CasRx, Cas13X, Cas13a, Cas13b, Cas13c, or Cas13Y. Even more preferably, the Cas enzyme is CasRx, Cas13X, or Cas13Y. Most preferably, the Cas enzyme is CasRx.

In some specific embodiments, the inhibitor is CRISPR gene editing tools, and the expression vector includes a nucleic acid encoding the Cas protein, a promoter for the Cas protein, a gRNA targeting the cell trans-differentiation factor, and a promoter for the gRNA. In some specific embodiments, the promoter for the Cas protein can be a broad-spectrum promoter or a specific promoter. The broad-spectrum promoter can be selected from CMV, CBH, CAG, PGK, SV40, EFIA, EFS, or pGlobin promoters, and the specific promoter can be a glial cell-specific promoter or a Muller glial (MG) cell-specific promoter. More preferably, the glial cell-specific promoter is selected from GFAP, ALDHILI, EAATI/GLAST, glutamine synthetase, S100ß, and EAAT2/GLT- 1 promoters, and the MG cell-specific promoter is selected from GFAP, ALDHILI, Glast (also known as Slcla3), and Rlbpl promoters. In some specific embodiments, the gRNA promoter can be operably linked to the U6 promoter.

In this invention, CasRx library screening cell line is first constructed through Tubb3-EGFP- CasRx-mESCs cell line. For the CasRx has the advantages of high efficiency and specificity, the screening library of RNA knocking achieved easily. The factors screened could influence non- neuronal cells such as stem cells and glial cells to produce neurons.

In some specific embodiments, the RNA-targeted CRISPR system CasRx is used to inhibit the gene or its RNA or its encoding protein.

As used in this article, Müller glial cells (MGs) are the major neuroglial cells in retinal tissue, while retinal ganglion cells (RGCs) are the innermost layer of neurons in the retina, with their dendrites primarily in contact with bipolar cells and their axons extending to the optic disc, forming the optic nerve.

In the present disclosure, the gene editor tools comprise DNA gene editor and/or RNA gene editor. In a preferred embodiment, the gene editor in the present disclosure comprises a gene editing protein and an optional gRNA.

Diseases related to neuronal function loss (also said neuronal dysfunction) or neuronal death

In this disclosure, unless otherwise stated, “function loss” and “dysfunction” have the same meaning, such as dopaminergic neuronal dysfunction also said dopaminergic neuronal function loss.

In this disclosure, diseases related to neuronal dysfunction or death mainly include those associated with dopaminergic neuronal dysfunction or death, as well as visual impairment associated with retinal ganglion cell (RGC) or photoreceptor loss or death.

In a preferred embodiment, diseases related to neuronal dysfunction or death include, but are not limited to: Parkinson's disease, schizophrenia, depression, visual impairment caused by RGC cell death, glaucoma, age-related RGC degeneration, optic nerve damage, retinal ischemia or hemorrhage, Leber's hereditary optic neuropathy; photoreceptor degeneration or death caused by injury or degenerative disease, macular degeneration, retinitis pigmentosa, diabetes-related blindness, night blindness, color blindness, inherited blindness, and congenital achromatopsia.

Astrocytes

Astrocytes are the most abundant type of cell in the mammalian brain. They perform many functions, including biochemical support (such as forming the blood-brain barrier), providing nutrients to neurons, maintaining extracellular ion balance, and participating in repair and scar formation after brain and spinal cord injury. Astrocytes can be divided into two types based on the content of their glial filament and the shape of their cytoplasmic processes: fibrous astrocytes, which are mostly distributed in the white matter of the brain and spinal cord, have long and thin processes with fewer branches and contain a large number of glial filament in the cytoplasm: and protoplasmic astrocytes, which are mostly distributed in the gray matter, have short and thick processes with more branches.

There is no particular restriction on the astrocytes that can be used in the present disclosure, including various types of astrocytes derived from the central nervous system of mammals, such as those derived from the striatum, ventral tegmental area, hypothalamus, spinal cord, dorsal midbrain, or cerebral cortex, preferably those derived from the striatum.

Neurons

In this disclosure, neurons refer to neurons that are capable of sending or receiving information through chemical or electrical signals. In some implementation schemes, functional neurons exhibit one or more functional characteristics of mature neurons present in the normal nervous system, including but not limited to excitability (e.g. the ability to exhibit action potentials, i.e. rapid depolarization followed by repolarization across the cell membrane voltage or membrane potential), formation of synaptic connections with other neurons, presynaptic neurotransmitter release, and postsynaptic responses (e.g. excitatory or inhibitory postsynaptic currents).

In some embodiments, the features of functional neurons include the expression of one or more markers of functional neurons, including but not limited to synaptic proteins, synaptophysin, glutamic acid decarboxylase 67 (GAD67), glutamic acid decarboxylase 65 (GAD65), parvalbumin, dopamine-and cAMP-regulated neuronal phosphoprotein 32 (DARPP32), vesicular glutamate transporter 1 (vGLUT1), vesicular glutamate transporter 2 (vGLUT2), acetylcholine, tyrosine hydroxylase (TH), dopamine, vesicular GABA transporter (VGAT), and gamma-aminobutyric acid (GABA).

Dopaminergic Neuron

Dopaminergic Neuron (dopamine neurons) contains and releases dopamine (DA) as neurotransmitter. Dopamine belongs to the neurotransmitter of catecholamines. It plays an important biological role in the central nervous system. The dopamine neurons in the brain are mainly concentrated in the substantria nigra pars sompacta of the middle brain, ventral tegmental area (VTA), hypothalamus and hypothalamus. Many trials certify that dopamine neurons are closely related to the various diseases of the human. The most typical disease is Parkinson's disease.

Gene Editor

In this disclosure, the gene editor refers to DNA gene editors and RNA gene editors. In one preferred embodiment, the gene editor disclosed herein includes a gene editing protein and an optional gRNA.

Gene Editing Protein

In this disclosure, the nucleotide sequence of the gene editing protein can be obtained by gene engineering techniques such as genome sequencing, polymerase chain reaction (PCR), etc., and its amino acid sequence can be deduced from the nucleotide sequence. The sources of the wild-type gene editing protein include (but are not limited to) Ruminococcus lavefaciens, Streptococcus pyogenes, Staphylococcus aureus, Acidaminococcus sp, and Lachnospiraceae bacterium.

In one preferred embodiment of this disclosure, the gene editing protein includes, but is not limited to, RNA-targeting gene editing proteins such as Cas13d, CasRx, Cas13X, Cas13a, Cas13b, Cas13c, and Cas13Y.

Protein and polynucleotide

“The term “protein”, “peptide”, and “polypeptide” are interchangeable in this disclosure and refer to the amino acid sequence of proteins or peptides selected from Plpp7, Fam126a, Gprc5c. Tmed4, Tle6, Psmd5, Mastl, Ssr3, Rhoa, Rfx8, Rbm10, Hnrnpa3, Prpf6, Pou3f3, Ncoal, Ccdc8, Adck1, Gjb2, Smad9, Nr2cl, Atp10b, Nidl, Tmcc3, Rad21, Amigol, Cep192, Sepp1, Klf12, Nxf1, Trp53inp2, Phlppl, Ptpdcl, Pebpl, Gm22174, Gm26117, Mir873a, Mir1900, Gm22414, Khdc4, Hnrnpa0, Hnrnph2, Srrm 1, Hnrnpf, Srsf4, Mbnl1, Zbtb42, Kcmf1, Gtf2i, Chgb, Fos, Kat2a, Tsg101, Hmgb4, Junb, Cdx2, Cers2, Rhox6, Thap3, and Zscan25, including the protein with or without an initiating methionine. In addition, the term also includes the full-length protein or its fragments. The protein referred to in this disclosure includes its full amino acid sequence, its secreted protein, its mutants, and its functionally active fragments.

In this disclosure, the terms “gene” and “nucleotide” can be used interchangeably, referring to the nucleotide sequences selected from Plpp7, Fam 126a, Gprc5c, Tmed4, Tlc6, Psmd5, Mastl, Ssr3, Rhoa, Rfx8, Rbm10, Hnrnpa3, Prpf6, Pou3f3, Ncoal, Ccdc8, Adckl, Gjb2, Smad9, Nr2cl, Atp10b, Nid1, Tmcc3, Rad21, Amigol, Cep192, Sepp1, Klf12, Nxf1, Trp53inp2, Phlppl, Ptpdcl, Pebpl, Gm22174, Gm26117, Mir873a, Mir1900, Gm22414, Khdc4, Hnrnpa0, Hnrnph2, Srrm1, Hnrnpf, Srsf4, Mbnl1, Zbtb42, Kcmfl, Gtf2i, Chgb, Fos, Kat2a, Tsg101, Hmgb4, Junb, Cdx2, Cers2, Rhox6, Thap3, and Zscan25.

In the presence of an amino acid fragment, the nucleotide sequence encoding it can be constructed and specific primers or probes can be designed based on the nucleotide sequence. The full-length nucleotide sequence or its fragment can usually be obtained by PCR amplification, recombination, or artificial synthesis. For PCR amplification, primers can be designed based on the nucleotide sequence disclosed in this disclosure, especially the open reading frame sequence, and cDNA libraries prepared by conventional methods known to those skilled in the art or commercially available cDNA libraries can be used as templates to amplify the relevant sequence. When the sequence is longer, multiple rounds of PCR amplification are often required, followed by correct assembly of the amplified fragments.

Once the relevant sequence is obtained, it can be obtained in large quantities by recombination. This is usually achieved by cloning it into a vector, transferring it into cells, and then isolating the relevant sequence from the proliferated host cells by conventional methods.

In addition, the relevant sequence can also be synthesized artificially, especially when the fragment length is shorter. Usually, a long fragment can be obtained by synthesizing multiple small fragments and then joining them together.

At present, the DNA sequence that encodes the protein (or its fragments, derivatives) in this invention can be obtained through chemical synthesis. The DNA sequence can then introduce the various existing DNA molecules (such as vectors) and cells known as the existing DNA molecules in the art.

Through conventional reorganization DNA technology, the polynucleotide sequence that can be used in this invention can be used to express or produce a polypeptide. Generally speaking, there are the following steps:

(1). Coded the polypeptide (or mutant) of the polypeptide in this invention, or use the reorganized expression carrier containing the polynucleotide to transform or transform the appropriate host cells:

(2). The host cells cultivated in the appropriate medium:

(3). Separate and purify protein from culture groups or cells.

In this disclosure, multi-nucleotide sequences can be inserted into recombinant expression vectors. As long as they can be replicated and maintained stably in the host, any plasmid and vector can be used. An important characteristic of an expression vector is that it usually contains a replication origin, promoter, selection marker, and translation control clements.

Techniques well known to those skilled in the art can be used to construct expression vectors containing the DNA sequences encoding Plpp7, Fam126a, Gprc5c, Tmed4, Tle6, Psmd5, Mastl, Ssr3, Rhoa, Rfx8, Rbm10, Hnrnpa3, Prpf6, Pou3f3, Ncoal, Ccdc8, Adck1, Gjb2, Smad9, Nr2el, Atp10b, Nidl, Tmcc3, Rad21, Amigol, Cep192, Sepp1, Klf12, Nxfl, Trp53inp2, Phlppl, Ptpdcl, Pebpl, Gm22174, Gm26117, Mir873a, Mir1900, Gm22414, Khdc4, Hnrnpa0, Hnrnph2, Srrm1, Hnrnpf, Srsf4, Mbnl1, Zbtb42, Kcmfl, Gtf2i, Chgb, Fos, Kat2a, Tsg101, Hmgb4, Junb, Cdx2, Cers2, Rhox6, Thap3, Zscan25 and appropriate transcription/translation control signals. These methods include in vitro DNA recombinant techniques, DNA synthesis techniques, codon optimization synthesis, and in vivo recombinant techniques. The DNA sequences can be effectively linked to appropriate promoters in the expression vector to guide mRNA synthesis. The expression vector also includes ribosome binding sites for translation initiation and transcription termination sites.

Furthermore, the expression vector preferably includes one or more selectable marker genes to provide a phenotypic characteristic for selecting host cells transformed, such as dihydrofolate reductase for eukaryotic cell culture, neomycin resistance, and green fluorescent protein (GFP), or tetracycline or ampicillin resistance for Escherichia coli.

A vector containing the appropriate DNA sequence and appropriate promoter or control sequence can be used to transform an appropriate host cell to allow it to express the protein.

Host cells can be prokaryotic cells, such as bacterial cells: either low-level eukaryotic cells, such as yeast cells: or high-level authentic cells, such as mammalian cells. Representative examples include: E. coli, bacterial cells of the genus of chains: fungal cells such as yeast: plant cells: insect cells: animal cells, etc.

The conventional technologies could be used to transformate of recombinant dna into host cells. When the host is a prokaryote, such as E. coli, the competent cells absorbing DNA could be harvested after exponential growth phase. If necessary; the electroporation also could be used. When the host cell is cucaryon, the following DNA transfection method can be selected: calcium phosphate precipitation,, conventional mechanical methods such as microinjection, electric perforation, liposome packaging, etc.

The transformer obtained can be cultivated in conventional methods to express the polypeptide encoded by the genes of this disclosure. According to the host cells used, the medium used in culture can be selected from various conventional medium. Cultivate under the conditions of the growth of host cells. When the host cells grow to the appropriate cell density, a promoter that induce the selection with the appropriate method (such as temperature conversion or chemical induction) will be cultivated for a period of time.

The reorganized polypeptide in the above method can be expressed in the cell, or on the cell membrane, or secreted to the outside of the cell. If it is necessary, it can be used to separate and purify the protein through various separation methods. These methods are well known by technicians in the art. Examples of these methods include, but not limited to: protein refolding, protein precipitation (salt fractionation), centrifugal, osmotic rupture of bacteria, super treatment, ultracentrifugation, molecular screen chromatography (gel filtering), adsorption chromatography, ion exchange chromatography; high performance liquid chromatography (HPLC) and other liquid chromatography:

Adeno-associated virus (AAV)

Adeno-associated virus (AAV) has received widespread attention as a gene therapy vector for inherited diseases due to its small size, lack of pathogenicity, and ability to transduce dividing and non-dividing cells.

AAV is a small, single-stranded DNA defective virus belonging to the family of dependoviruses, which requires a helper virus (usually adenovirus) for replication. It encodes the cap and rep genes in the two terminal inverted repeats (ITRs) of the virus, which are crucial for viral replication and packaging. The cap gene encodes the viral capsid protein, and the rep gene participates in viral replication and integration. AAV can infect a variety of cells.

Recombinant AAV vectors (rAAVs) are derived from nonpathogenic wild-type AAV and are considered one of the most promising gene transfer vectors due to their good safety profile, broad host cell range (dividing and non-dividing cells), low immunogenicity; and long-term expression of exogenous genes in vivo. After more than a decade of research, the biological characteristics of rAAV have been extensively studied, and much data has been accumulated, particularly regarding its application in various cells, tissues, and in vivo experiments. In medical research, rAAV has been used in gene therapy studies for various diseases (including in vitro and in vivo experiments), and as a distinctive gene transfer vector, it has also been widely used in gene function studies, disease model construction, and gene knockout mice preparation.

In an exemplary embodiment of the present disclosure, the vector is a recombinant AAV vector. AAV is a relatively small DNA virus that can stably and site-specifically integrate into the genome of the cells they infect. They can infect a wide range of cells without affecting cell growth, morphology; or differentiation, and do not appear to be involved in human pathology: The AAV genome has been cloned, sequenced, and characterized. AAV contains inverted terminal repeat (ITR) regions of approximately 145 base pairs at each end, which serve as the replication initiation point for the virus. The rest of the genome is divided into two important regions with capsid-forming functions: the left portion of the genome, which contains the rep gene involved in virus replication and gene expression, and the right portion of the genome, which contains the cap gene encoding the viral capsid protein.

The AAV vector can be prepared using standard methods known in the art. Any serotype of adeno-associated virus is suitable. Methods for purifying the vector can be found, for example, in U.S. Pat. No. 6,566,118, 6989264, and 6995006, the contents of which are incorporated herein by reference in their entirety. The preparation of hybrid vectors is described in, for example, PCT Application No. PCT/US2005/027091, the contents of which are incorporated herein by reference in their entirety: The use of AAV-derived vectors for the in vitro and in vivo delivery of genes is well described (sec, for example, International Patent Application Publications No. WO91/18088 and WO93/09239: U.S. Pat. No. 4,797,368, 6,596,535, and 5,139,941; and European Patent No. 0488528, the contents of which are incorporated herein by reference in their entirety). These patent publications describe various AAV-derived constructs where the rep and/or cap genes are missing and have been replaced with the gene of interest, and the use of these constructs for the delivery of the gene of interest either in vitro (into cultured cells) or in vivo (directly into an organism). Replication-defective recombinant AAV can be prepared by co-transfecting into a human helper virus (such as adenovirus) infected cell line: a plasmid containing the gene of interest flanked on either side by two AAV inverted terminal repeat (ITR) regions, and a plasmid carrying the AAV capsid genes (rep and cap genes). The resulting AAV recombinants are then purified by standard techniques.

In some embodiments, the recombinant vector is packaged into viral particles (such as AAVI, AAV2, AAV3, AAV4, AAV5, AAV6, AAV7, AAV8, AAV9, AAV10, AAV11, AAV12, AAV13, AAV14, AAV15, and AAV16 AAV viral particles). Thus, the present disclosure includes recombinant virus particles containing any vector described herein (as they contain a recombinant nucleic acid). Methods for producing such particles are known in the art and are described, for example, in U.S. Pat. No. 6,596,535.

Inhibitor and pharmaceutical composition

By using the protein disclosed in this invention and various conventional screening methods, substances that interact with Plpp7, Fam126a, Gprc5c, Tmed4, Tle6, Psmd5, Mastl, Ssr3, Rhoa, Rfx8, Rbm10, Hnrnpa3, Prpf6, Pou3f3, Ncoal, Ccdc8, Adck1, Gjb2, Smad9, Nr2cl, Atp10b, Nidl, Tmcc3, Rad21, Amigol, Cep192, Seppl, Klf12, Nxfl, Trp53inp2, Phlppl, Ptpdcl, Pebpl, Gm22174, Gm26117, Mir873a, Mir1900, Gm22414, Khdc4, Hnrnpa0, Hnrnph2, Srrm1, Hnrnpf, Srsf4, Mbnl1, Zbtb42, Kcmf1, Gtf2i, Chgb, Fos, Kat2a, Tsg101, Hmgb4, Junb, Cdx2, Cers2, Rhox6, Thap3, Zscan25 genes or proteins can be screened, especially inhibitors, and other substances that interact with them.

In this disclosure, inhibitors (or antagonists) can be used to reduce or eliminate the expression and/or activity of the gene, its RNA (such as mRNA), or the encoded protein at the DNA, RNA, or protein level.

For example, the inhibitors include antibodies, antisense RNA, siRNA, shRNA, miRNA, gene editors, Protac technology; epigenetic regulatory elements, or active inhibitors. A preferred inhibitor is a gene editor that can suppress expression.

In one preferred embodiment, the method and steps of inhibition include using an antibody to neutralize the protein, using a virus (such as an adenovirus) carrying shRNA or siRNA or a gene editor to silence the gene.

The inhibition rate is generally at least 50% or more, preferably 60%, 70%, 80%, 90%, or 95% or more inhibition, which can be controlled and detected based on conventional techniques such as flow cytometry, fluorescence quantitative PCR, or Western blot.

The inhibitors of the present disclosure (including antibodies, antisense nucleic acids, gene editors, and other inhibitors) when administered for treatment can inhibit the expression and/or activity of the protein, thereby inducing glial cells to differentiate into neuronal cells and treating diseases related to neuronal dysfunction or death. Typically, these substances can be formulated in non-toxic, inert, and pharmaceutically acceptable aqucous carrier media, with pH usually around 5- 8, preferably around 6-8, although pH values can vary depending on the nature of the formulated substance and the disease being treated. The formulated drug combinations can be administered by conventional methods, including (but not limited to): local, intramuscular, intracranial, intraocular, intraperitoneal, intravenous, subcutancous, intradermal, topical administration, and autologous cell extraction and culturing followed by reinfusion.

This disclosure also provides a pharmaceutical composition comprising a safe and effective amount of the disclosed inhibitor (such as an antibody, gene editor, antisense sequence such as siRNA) and a pharmaceutically acceptable carrier or excipient. Such carriers include (but are not limited to): saline, buffer, glucose, water, glycerol, ethanol, and combinations thereof. The pharmaceutical formulation should be matched with the route of administration. The pharmaceutical composition of this disclosure can be formulated into injectable form, for example, prepared by conventional methods with saline or glucose and other excipients. Pharmaceutical compositions such as tablets and capsules can be prepared by conventional methods. The pharmaceutical compositions such as injectables, solutions, tablets and capsules should be manufactured under aseptic conditions. The dosage of the active ingredient is an effective amount for treatment, such as about 1 ug to 10 mg per kilogram of body weight per day:

The main advantages of the present disclosure include:

(1) The present disclosure discovered for the first time that reducing the expression or activity of Plpp7. Fam 126a, Gprc5c, Tmed4, Tlc6, Psmd5, Mastl, Ssr3, Rhoa, Rfx8, Rbm10, Hnrnpa3, Prpf6, Pou3f3, Ncoal, Ccdc8, Adck1, Gjb2, Smad9, Nr2cl, Atp10b, Nidl, Tmcc3, Rad21, Amigol, Cep192, Scpp1, Klf12, Nxfl, Trp53inp2, Phlpp1, Ptpdc1, Pebpl, Gm22174, Gm26117, Mir873a. Mir1900, Gm22414, Khdc4, Hnrnpa0, Hnrnph2, Srrm1, Hnrnpf, Srsf4, Mbnll, Zbtb42, Kcmfl, Gtf2i, Chgb, Fos, Kat2a, Tsg 101, Hmgb4, Junb, Cdx2, Cers2, Rhox6, Thap3, Zscan25 genes or their encoded proteins in non-neuronal cells can induce the differentiation of non-neuronal cells into neurons and/or neural precursor cells, thereby preventing and/or treating diseases related to neuronal dysfunction or death.

(2) The present disclosure discovered for the first time that using gene editing tools (including gene editing proteins and gRNA) to inhibit the expression of Plpp7, Fam 126a, Gprc5c. Tmed4, Tle6, Psmd5, Mastl, Ssr3, Rhoa, Rfx8, Rbm10, Hnrnpa3, Prpf6, Pou3f3, Ncoal, Ccdc8, Adckl, Gjb2, Smad9, Nr2cl, Atp10b, Nidl, Tmcc3, Rad21, Amigol, Cep192, Sepp1, Klf12. Nxfl. Trp53inp2, Phlpp1, Ptpdcl, Pcbpl, Gm22174, Gm26117, Mir873a, Mir1900, Gm22414, Khdc4, Hnrnpa0.

Hnrnph2, Srrm1, Hnmnpf, Srsf4, Mbnl1, Zbtb42, Kcmfl, Gtf2i, Chgb, Fos, Kat2a, Tsg 101, Hmgb4, Junb, Cdx2, Cers2, Rhox6, Thap3, Zscan25 in non-neuronal cells such as glial cells can induce the conversion of non-neuronal cells (such as stem cells, neural precursor cells, or glial cells) into neurons and/or neural precursor cells, providing a potential approach for the treatment of diseases related to neuronal dysfunction or death.

(3) Transplantation of induced neural progenitor cells or dopamine progenitor cells, which differentiate/convert to dopamine neurons, alleviates motor function disorders in a mouse model of Parkinson's disease.

(4) The RNA-targeting CRISPR system CasRx can avoid the risk of permanent DNA changes caused by traditional CRISPR-Cas9 editing. Therefore, CasRx-mediated RNA editing provides an effective means of treating various diseases.

(5) This disclosure describes the direct conversion of Müller glia cells into retinal ganglion cells or photoreceptor cells by suppressing the expression of Plpp7, Fam 126a, Gprc5c, Tmed4, Tle6, Psmd5, Mastl, Ssr3, Rhoa, Rfx8, Rbm10, Hnrnpa3, Prpf6, Pou3f3, Ncoal, Ccdc8, Adckl, Gjb2, Smad9, Nr2el, Atp10b, Nidl, Tmcc3, Rad21, Amigol, Cep192, Sepp1, Klf12, Nxfl, Trp53inp2, Phlpp1, Ptpdcl, Pebpl, Gm22174, Gm26117, Mir873a, Mir1900, Gm22414, Khdc4, Hnrnpa0, Hnrnph2, Srrm1, Hnrnpf, Srsf4, Mbnl1, Zbtb42, Kcmfl, Gtf2i, Chgb, Fos, Kat2a, Tsg101, Hmgb4, Junb, Cdx2, Cers2, Rhox6, Thap3, and Zscan25.

(6) Regenerated retinal ganglion cells or photoreceptor cells can be integrated into the visual pathway and improve visual function in mouse models of RGC or photoreceptor cell damage.

(7) This disclosure provides an excellent tool for treating various diseases by using the RNA- targeting CRISPR system CasRx to knock down Plpp7, Fam126a, Gprc5c, Tmed4, Tle6, Psmd5, Mastl, Ssr3, Rhoa, Rfx8, Rbm10, Hnrnpa3, Prpf6, Pou3f3, Ncoal, Ccdc8, Adckl, Gjb2, Smad9, Nr2cl, Atp10b, Nidl, Tmcc3, Rad21, Amigol, Cep192, Sepp1, Klf12, Nxfl, Trp53inp2, Phlppl, Ptpdc1, Pebpl, Gm22174, Gm26117, Mir873a, Mir1900, Gm22414, Khdc4, Hnrnpa0, Hnrnph2, Srrm1, Hnrnpf, Srsf4, Mbnl1, Zbtb42, Kcmfl, Gtf2i, Chgb, Fos, Kat2a, Tsg101, Hmgb4, Junb, Cdx2, Cers2, Rhox6, Thap3, and Zscan25.

Further explanation of the present disclosure will be provided through specific examples. It should be understood that these examples are only intended to illustrate the present disclosure and not to limit the scope of the present disclosure. Experimental methods in the examples that do not specify specific conditions are generally carried out under conventional conditions, such as those described in Molecular Cloning: A Laboratory Manual (New York: Cold Spring Harbor Laboratory Press, 1989) by Sambrook et al., or under conditions recommended by the manufacturer. Unless otherwise specified, percentages and quantities are weight percentages and weight quantities. Unless otherwise specified, materials and reagents used in the examples of the present disclosure are commercially available products.

General methods

Animal ethics

The use and care of animals in this study comply with the guiding principles of the Biomedical Research Ethics Committee of the Center for Excellence in Brain Science and Intelligence

Technology of the Chinese Academy of Sciences.

Construction of lentiviral sgRNA library

To construct the lentiviral sgRNA library; the gRNA library was first amplified by PCR using Q5 high-fidelity enzyme, and the PCR products were recovered by agarose gel electrophoresis and then ligated into the Lenti-Cre lentiviral vector. The ligation products were recovered with sodium acetate and then electroporated using a Bio-Rad electroporator with competent cells purchased from Weidi Biological. The electroporation products were transferred to 5 ml of SOC medium without antibiotics, and revived at 37° C. and 200 rpm for 1 h, and then excess medium was removed by centrifugation and an appropriate amount of cells were plated. The plate was incubated overnight at 37° C., and the next day the colonies were scraped off and plasmids were extracted for lentiviral packaging. The packaged lentivirus is the desired lentiviral library; named Lenti-gRNAs-Cre.

Cultivation of mouse embryonic stem cells (mESCs):

mESCs are cultured in stem cell medium consisting of DMEM (Milipore), FBS (Gibco), NEAA (Miliporc), PS (Gibco), L-Glutamine (Miliporc), ß-mercaptocthanol, PD0325901, CHIR99021, and mouse LIF (Milipore). mESCs have a fast growth rate and are passaged every two days.

Establishment of mouse embryonic stem cell (mESC) line:

To establish a stable mESC line expressing CAG-LSL-CasRx, mESCs were passaged into a 6- well plate and transfected within 24 hours using lipo3000 with a mixture of CAG-LSL-CasRx plasmid and U6-sgRNA-CBH-Cas9 plasmid. The medium was changed 12 hours after transfection. On day 3 after transfection, the cells were passaged, and on day 7, the cells were sorted by flow cytometry and single cells were deposited into a 96-well plate. After clonal expansion, the cells were subjected to PCR and sequencing to identify positive clones. The correct clones were named CasRx- mESCs for downstream experiments.

To establish a TUBB3-GFP cell line on the CasRx-mESCs line, CasRx-mESCs were transfected with TUBB3-GFP plasmid and U6-sgRNA-CBH-Cas9 plasmid using lipo3000 and the medium was changed 12 hours after transfection. Two days after transfection, green fluorescent cells were sorted by flow cytometry and deposited into a 12-well plate for 7 days of culture. Then, non-fluorescent single cells were sorted by flow cytometry and deposited into a 96-well plate. Positive clones were identified and named TUBB3-GFP-CasRx-mESCs for subsequent screening experiments.

Library screening and induction differentiation

To induce cell differentiation, Gelatin-coated cell culture dishes were first prepared (37° C., 30 min). Digest the cells and transfer them to the prepared culture dishes, using mESCs basic culture medium. After 12 hours of passaging, switch to differentiation medium and add Lenti-gRNAs-Cre library with Polybrene (final concentration of 10 ug/ml). After 24 hours of incubation, replace with fresh differentiation medium and add Puromycin (final concentration of 1 ug/ml). Replace with fresh differentiation medium every 48 hours thereafter. After 12 days of induction, use flow cytometry to sort for EGFP-positive cells (Tubb3-EGFP), PCR amplify the U6-gRNA universal sequence, and analyze the enriched gRNA sequences by high-throughput sequencing. In individual verification experiments, TUBB3-GFP-CasRx-mESCs cells were cultured in a 6-well plate, sequentially added with Lenti-gRNA-Cre, and cultured in N2B27 induction medium for differentiation, for use in flow cytometry or immunofluorescence staining experiments.

Lentivirus Package

In order to achieve long-term infection of mouse embryonic stem cells (mESCs), we chose to package gRNA using lentivirus for cell infection. Lentivirus packaging was carried out using 293T cells in the P2 laboratory. When the cell confluence reached 70-90%, transfection was carried out using PEI (Shanghai Liji Biotechnology Co., Ltd.), and the plasmids transfected were the target plasmids (library), sPAX2 (packaging plasmid), and Pmd2.G (envelope plasmid). After 6-10 hours of transfection, fresh culture medium was replaced, and the virus was harvested after 60 hours. The virus supernatant was filtered using a 0.22 um filter membrane to remove cell debris, and the supernatant was collected and subjected to ultra-high-speed centrifugation using an ultracentrifuge. The supernatant was then discarded, and the lentivirus precipitate was dissolved in DPBS. The packaged lentivirus is the target lentivirus library, named Lenti-gRNAs-Cre.

Flow analysis

Cell digestion was performed in a super clean bench. The supernatant was sucked away using a vacuum pump, an appropriate amount of DPBS was added to wash the cells, and then an appropriate amount of 0.05% trypsin was added. After 2 minutes of digestion in the cell culture incubator, an equal amount of cell culture medium was added to terminate the digestion. The cells were transferred to a 1.5 ml EP tube and centrifuged at 1000 rpm for 3 minutes to remove the supernatant. An appropriate amount of cell culture medium was added to resuspend the cells. In the flow cytometry sorting experiment (BD fusion or Beckman XDP), an appropriate amount of medium was added to the collection tube. After collection, the cells were used for further culture or downstream analysis. In the flow cytometry analysis experiment (BD LSRFortessaX-20), a total of 20,000 valid cells were collected and the proportion of EGFP-positive cells (Tubb3-EGFP) was analyzed.

Cellular immunofluorescence staining and imaging

To verify the differentiation effect of different gRNAs added, cells induced to differentiate by Lenti-gRNA-Cre were fixed with 4% PFA for 10 minutes and washed with PBS (5 min x 4 times) after fixation. Primary antibodies were added (overnight at 4° C. or 2-3h at room temperature) and then fluorescent secondary antibodies were used for staining (2-3h at room temperature). The primary antibody used in this study was rabbit anti-Map2 (Cell Signaling Technology, 4542S, 1:1000), and the secondary antibody used was CyTM5 AffiniPure Donkey Anti-Rabbit IgG (H+L) (Jackson ImmunoResearch, 711-175-152, 1:500). Imaging was performed on an FV3000 (Olympus) confocal microscope. Cells cultured in cell culture dishes were imaged using a regular fluorescence microscope.

AAV packaging and injection

To overexpress CasRx in glial cells, the glial cell-specific promoter GFAP was used to drive CasRx expression, and gRNA was expressed by the U6 promoter. The above expression elements were assembled onto an AAV vector using In-Fusion cloning for AAV packaging. AAV packaging was performed using 293T cells, which were transfected with a 3-plasmid system, and virus was collected and purified by ultracentrifugation. The purified AAV was used for in vivo AAV injection in mice, and AAV8 and AAV9 scrotypes were used in this study: The control group was AAV-CasRx +mCherry, and the experimental group was AAV-CasRx-gRNA +mCherry. Different groups of AAV were injected into the striatum of mice (AP +0.8 mm, ML +1.6 mm, and DV-2.8 mm), and analysis was performed approximately 1-2 months after injection.

Mouse tissue immunofluorescence staining

Tissues were harvested and sliced after 1.5-2 months of injection, and immunofluorescence staining was performed. Mice were perfused with saline and 4% PFA, and the brain was removed and fixed with 4% paraformaldehyde (PFA) overnight or at room temperature for 12 hours. The tissue was then dehydrated in 30% sucrose for at least 12 hours and embedded in OCT. Frozen sections were cut at a thickness of 30 um or 40 um on a Thermo freezing microtome. For immunofluorescence staining, brain slices were washed three times with 0.1M phosphate-buffered saline (PBS) for 5-10 minutes each time. After overnight incubation with primary antibody (4° C.), the slices were washed with PBS (10 min, 3-4 times) and then incubated with secondary antibody (2-3 hours at room temperature). After incubation, the slices were washed with PBS 3-4 times (10 minutes each time). Finally, the slices were sealed with an anti-fluorescence quench sealing tablets (Life Technology) for storage.

Intravitreal injection and Subretinal injection

In order to conduct NMDA modeling, a 200 mM NMDA solution was prepared in PBS, and then 1.5 ul of the solution was injected into the vitreous body of a mouse using a glass micropipette after the mouse was anesthetized. For AAV injection, 1 ul of GFAP-GFP-Cre (0.1 ul) +PBS (0.9 ul) or GFAP-GFP-Cre (0.1 ul) +U6-gRNA-GFAP-CasRx (0.9 ul) was injected into the subretinal space of Ai9 micc.

Immunofluorescent staining

After 1-2 months of AAV injection, eyes and optic nerves were collected and fixed with 4% PFA for about 2 hours, dehydrated in a 30% sucrose solution (for eyes) or directly embedded in OCT and frozen. Eye slices (30 um) were used for immunofluorescence staining. The primary antibodies used were mouse anti-Brn3a (1:100, MAB1585, Millipore) and rabbit anti-RBPMS (1:500, 15187-1-AP, Proteintech), and the secondary antibodies were CyTM5 AffiniPure Donkey anti-mouse IgG (H+L) (1:500, 715-175-150, Jackson ImmunoResearch) and CyTM5 AffiniPure Donkey anti-rabbit IgG (H+L) (1:500, 711-175-152, Jackson ImmunoResearch). After antibody incubation, the slices were washed with PBS and mounted with an anti-fade reagent before being imaged under an Olympus FV3000 microscope.

EXAMPLES

Example 1

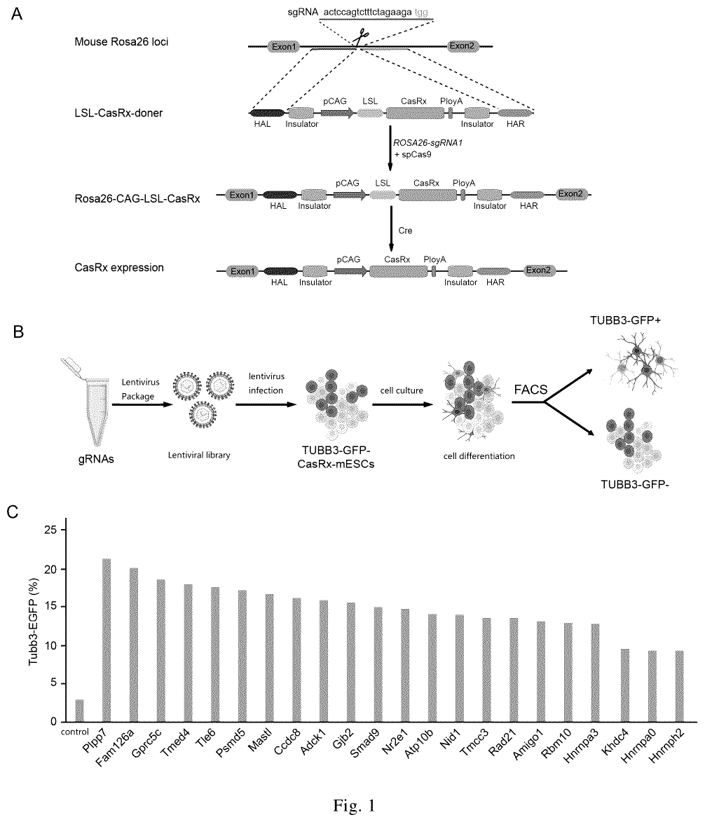

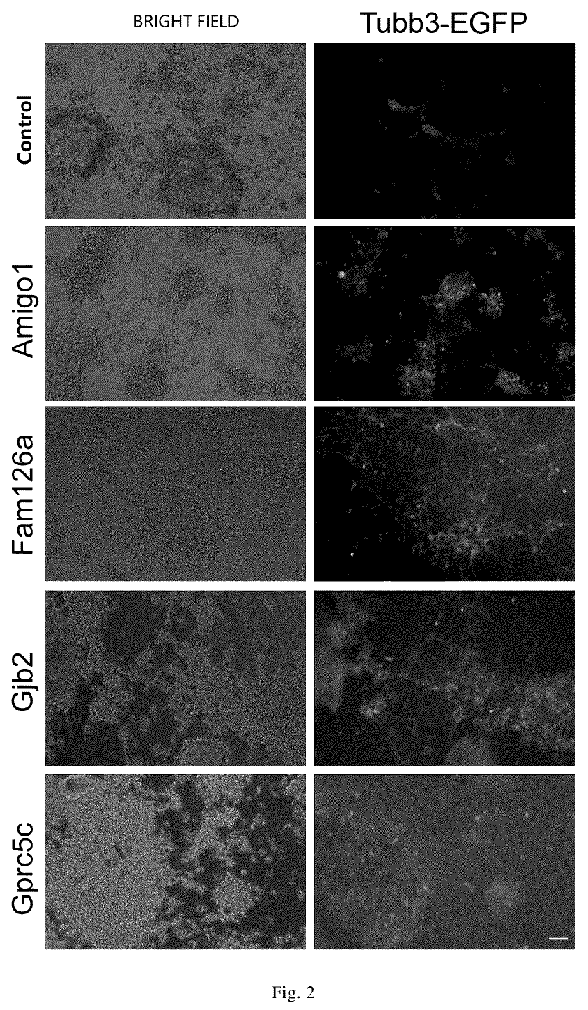

To screen for neural conversion factors, we first constructed a CAG-LSL-CasRx mouse embryonic stem cell line (CasRx-mESCs), and further constructed a Tubb3-EGFP-CasRx-mESC cell line by knocking in an EGFP fluorescent reporting system at the Tubb3 locus. When Tubb3- EGFP-CasRx-mESCs differentiate into neurons, Tubb3 activates EGFP expression. By analyzing factors that are highly expressed in glial cells but low in neurons, we designed three gRNAs for cach gene and constructed them into the Lenti-Cre backbone vector, which is the target lentiviral library Lenti-gRNAs-Cre. In this study, we used the Lenti-gRNAs-Cre library to infect Tubb3-EGFP- CasRx-mESC cells and induced differentiation in induction medium, and enriched factors that promote mESCs differentiation into neurons through flow sorting (FIG. 1A). Each factor was then individually constructed into a lentiviral vector and used to infect Tubb3-EGFP-CasRx-mESC cell lines and induce differentiation (FIG. 1B). Flow cytometry analysis results showed that in the control group that did not target any sequence, the average percentage of Tubb3-EGFP-positive cells was approximately 2.96%, while in the groups containing gRNAs targeting different factors, the percentage of Tubb3-EGFP-positive cells increased significantly. Among them, the Tubb3-EGFP- positive cell proportion of the group targeting Plpp7, Fam126a, Gprc5c, Tmed4, Tlc6, Psmd5, Mastl, Ccdc8, Adck1, Gjb2, Smad9, Nr2el, Atp10b, Nid1, Tmcc3, Rad21, Amigo1, Rbm 10, Hnrnpa3, and other factor groups exceeded 12% (FIG. 1C). To further study the function of these factors in promoting non-neuronal cells to produce neurons, we targeted genes such as Amigo1, Fam126a, Gjb2, and Gprc5c with Lenti-gRNA-Cre and imaged them under a fluorescence microscope. In the control group, only a very small number of green fluorescent cells were found, while in the experimental groups targeting Amigol, Fam126a, Gjb2, and Gprc5c, mESCs clearly underwent differentiation. Compared with the control group, the number of EGFP-positive cells increased significantly; and most of these EGFP-positive cells had typical neuronal morphological characteristics (FIG. 2). The above results indicate that these factors can promote non-neuronal cells to produce neurons.

Example 2

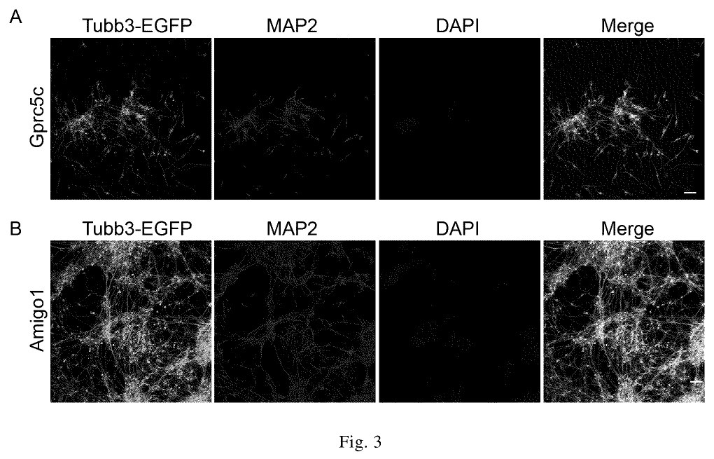

In order to further investigate whether the screened factors can promote the generation of mature neurons from non-neuronal cells (mESCs), this study used the mature neuronal protein marker (MAP2) for immunofluorescence staining. The results showed that after targeting Amigol and Gprc5c with CasRx and inducing differentiation, there were many cells expressing green fluorescence, and MAP2 staining also showed that most of these cells expressed MAP2 (FIG. 3A and 3B). This indicates that the factors screened out through the Lenti-gRNAs-Cre library in the Tubb3- EGFP-CasRx-mESCs system can efficiently promote the generation of neurons from non-neuronal cells (mESCs), and can partially mature into MAP2-positive neurons.

Example 3

To further investigate whether the factors selected in this study can induce glial cells to differentiate into neurons in vivo, we constructed an AAV-U6-gRNA-GFAP-CasRx system targeting the factors obtained from the screening.

REFERENCES 1. Mccarthy, K.D. & Devellis, J. Preparation Of Separate Astroglial And Oligodendroglial Cell- Cultures From Rat Cerebral Tissue. Journal Of Cell Biology 85, 890-902 (1980).

2. Zhou, H. et al. Cerebellar modules operate at different frequencies. Elife 3, c02536 (2014).

3. Xu, H.T. et al. Distinct lineage-dependent structural and functional organization of the hippocampus. Cell 157, 1552-1564 (2014).

4. Su, J. et al. Reduction of HIP2 expression causes motor function impairment and increased vulnerability to dopaminergic degeneration in Parkinson's disease models. Cell Death Dis 9, 1020 (2018).

5. Chavez, A. et al. Comparison of Cas9 activators in multiple species. Nat Methods 13, 563-567 (2016).

6. Qian, Hao et al. Reversing a model of Parkinson's disease with in situ converted nigral neurons. Nature 582, 550-556 (2020).

7. Zhou, Haibo et al. Glia-to-Neuron Conversion by CRISPR-CasRx Alleviates Symptoms of Neurological Disease in Mice. Cell 181,590-603.e16 (2020).

All the references mentioned in this disclosure are cited as references in this application, just as each reference is cited individually as a reference. Additionally, it should be understood that, after reading the teachings of this disclosure as described above, those skilled in the art may make various changes or modifications to this disclosure, which are considered to be equivalent forms falling within the scope of the claims attached to this application.

1. A method for converting non-neuronal cells into neurons or neural progenitor cells, characterized in that the method comprises reducing the expression or activity of a cell trans-differentiation factor, wherein the cell trans-differentiation factor is at least one gene, or at least one gene's RNA, or at least one gene-encoded protein selected from Plpp7, Fam126a, Gprc5c, Tmed4, Tle6, Psmd5, Mast1, Ssr3, Rhoa, Rfx8, Rbm10, Hnrnpa3, Prpf6, Pou3f3, Ncoa1, Ccdc8, Adck1, Gjb2, Smad9, Nr2e1, Atp10b, Nid1, Tmcc3, Rad21, Amigo1, Cep192, Sepp1, Klf12, Nxf1, Trp53inp2, Phlpp1, Ptpdc1, Pebp1, Gm22174, Gm26117, Mir873a, Mir1900, Gm22414, Khdc4, Hnrnpa0, Hnrnph2, Srrm1, Hnrnpf, Srsf4, Mbn11, Zbtb42, Kcmf1, Gtf2i, Chgb, Fos, Kat2a, Tsg101, Hmgb4, Junb, Cdx2, Cers2, Rhox6, Thap3, or Zscan25;
preferably, the non-neuronal cells are selected from stem cells, progenitor cells or terminally differentiated cells;
more preferably, the non-neuronal cells are selected from non-neuronal cells of mammals, such as humans, non-human primates, mice, rats;
even more preferably, the non-neuronal cells of mammals are selected from the stem cells or terminally differentiated cells;
even more preferably, the differentiation efficiency of non-neuronal cells is at least 0.1%, 1%, 10%, 20%, 30%, 40%, 50%, 60%, or higher.

2. The method according to claim 1, wherein the stem cells are embryonic stem cells, neural stem cells, or induced pluripotent stem cells.

3. The method according to claim 1, wherein the terminally differentiated cells are glial cells; preferably, the glial cells are selected from astrocytes, oligodendrocytes, microglia, NG2 cells, Müller glia cells, glioblastoma cells, or spiral ganglion glia cells; more preferably, the glial cells are selected from astrocytes, Müller glia cells or spiral ganglion glia cells.

4. The method according to claim 1, characterized in that non-neuronal cells are cultured in vitro and the expression or activity of cell trans-differentiation factors are reduced, so that non-neuronal cells are transformed into neurons or neural precursor cells in vitro; or non-neuronal cells in vivo are induced to transform into neurons or neural precursor cells by reducing the expression or activity of cell trans-differentiation factors in vivo; preferably, the non-neuronal cells are glial cells, which are transformed into neurons or neural precursor cells in vivo.

5. The method according to any one of claims 1-4, characterized in that the method of reducing the expression or activity of cell trans-differentiation factors includes administering an inhibitory substance that reduces the expression or activity of cell trans- differentiation factors; the inhibitory substance includes an inhibitor of the expression or activity of at least one gene, RNA, or protein encoding at least one gene selected from Plpp7, Fam126a, Gprc5c, Tmed4, Tle6, Psmd5, Mast1, Ssr3, Rhoa, Rfx8, Rbm10, Hnrnpa3, Prpf6, Pou3f3, Ncoa1, Ccdc8, Adck1, Gjb2, Smad9, Nr2e1, Atp10b, Nid1, Tmcc3, Rad21, Amigo1, Cep192, Sepp1, Klf12, Nxf1, Trp53inp2, Phlpp1, Ptpdc1, Pebp1, Gm22174, Gm26117, Mir873a, Mir1900, Gm22414, Khdc4, Hnrnpa0, Hnrnph2, Srrm1, Hnrnpf, Srsf4, Mbn11, Zbtb42, Kcmf1, Gtf2i, Chgb, Fos, Kat2a, Tsg101, Hmgb4, Junb, Cdx2, Cers2, Rhox6, Thap3, or Zscan25;
preferably, the inhibitor is selected from gene editing tools that regulate the expression of cell trans-differentiation factors, epigenetic regulation tools, antibodies, small molecule compounds, mRNA, microRNA, siRNA, shRNA, antisense oligonucleotides, binding proteins or protein domains, peptides, nucleic acid aptamers, PROTACs, expression vectors containing promoters, protein analogs, artificially synthesized or modified inhibitors, or combinations thereof;
more preferably, the gene editing tool includes: (a) a gene editing system or its expression vector, wherein the gene editing system is selected from the CRISPR system (including the CRISPR/dCas system), the ZFN system, the TALEN system, the RNA editing system, or combinations thereof; and/or (b) one or more required gRNAs or their expression vectors, wherein the gRNA guides gene editing proteins to specifically bind to the DNA or RNA of the gene.

6. The method according to claim 5, wherein the CRISPR system is used to reduce the expression or activity of cell trans-differentiation factors; preferably, the CRISPR gene editing tool includes an encoding nucleic acid for a Cas enzyme or a functional domain of a Cas enzyme, as well as a gRNA targeting the cell trans-differentiation factors; more preferably, the Cas enzyme is selected from Cas13d, CasRx, Cas13X, Cas13a, Cas13b, Cas13c, or Cas13Y, and even more preferably, the Cas enzyme is CasRx.

7. The method according to claim 5, wherein the inhibitory substance further comprises a carrier; preferably, the carrier is a viral vector, lipid nanoparticles (LNP), liposomes, cationic polymers (such as PEI), nanoparticles, exosomes, or virus-like particles; more preferably, the carrier is an AAV vector or lipid nanoparticles.

8. The method according to claim 3, wherein the astrocytes are derived from the brain or spinal cord, the Müller glia cells are derived from the retina, or the spiral ganglion glia cells are derived from the inner ear or vestibulum; preferably, the brain is select from the cerebrum, midbrain, cerebellum, or brainstem, and more preferably, from the striatum or substantia nigra.

9. The method according to claim 1, wherein the neuronal cells are mammalian neurons, such as human, non-human primate, rat, or mouse neurons; preferably, the neuronal cells are dopamine neurons, 5-HT neurons, NE neurons, ChAT neurons, GABA neurons, glutamatergic neurons, motor neurons, photoreceptor cells (such as rod cells and cone cells), retinal ganglion cells (RGC), cochlear nerve cells (such as spiral ganglion neurons and vestibular neurons), or medium spiny neurons (MSN), or a combination thereof; more preferably, the neuronal cells are dopamine neurons, retinal ganglion cells, and photoreceptor cells;
preferably, the non-neuronal cells are astrocytes, and the neuronal cells are dopamine neurons. alternatively, the glial cells are Müller glia cells, and the neuronal cells are RGC or photoreceptor cells.

10. The method according to claim 1, characterized in that the cell trans-differentiation factors are selected from at least one gene, or at least one RNA, or at least one protein encoded by a gene, selected from Plpp7, Fam126a, Gprc5c, Tmed4, Tle6, Psmd5, Mastl, Ccdc8, Adck1, Gjb2, Smad9, Nr2e1, Atp10b, Nid1, Tmcc3, Rad21, Amigol, Rbm10, Hnrnpa3; preferably, the cell trans-differentiation factor is selected from at least one gene, or at least one RNA, or at least one protein encoded by a gene, selected from Amigo1, Fam126a, Gjb2, or Gprc5c.

11. A method for the prevention or treatment of disease associated with neuronal dysfunction or death, comprising administering an inhibitor that reduces the expression or activity of a cell trans-differentiation factor to required subjects, said trans-differentiation factor is selected from at least one gene, or at least one RNA, or at least one protein encoded by the gene, selected from Plpp7, Fam126a, Gprc5c, Tmed4, Tle6, Psmd5, Mast1, Ssr3, Rhoa, Rfx8, Rbm10, Hnrnpa3, Prpf6, Pou3f3, Ncoa1, Ccdc8, Adck1, Gjb2, Smad9, Nr2e1, Atp10b, Nid1, Tmcc3, Rad21, Amigo1, Cep192, Sepp1, Klf12, Nxf1, Trp53inp2, Ph1pp1, Ptpdc1, Pebp1, Gm22174, Gm26117, Mir873a, Mir1900, Gm22414, Khdc4, Hnrnpa0, Hnrnph2, Srrm1, Hnrnpf, Srsf4, Mbn11, Zbtb42, Kcmf1, Gtf2i, Chgb, Fos, Kat2a, Tsg101, Hmgb4, Junb, Cdx2, Cers2, Rhox6, Thap3, Zscan25; preferably, the cell trans bi-differentiation factor is selected from at least one gene, or at least one RNA, or at least one protein encoded by the gene, selected from Plpp7, Fam126a, Gprc5c, Tmed4, T1e6, Psmd5, Mast1, Ccdc8, Adck1, Gjb2, Smad9, Nr2e1, Atp10b, Nid1, Tmcc3, Rad21, Amigo1, Rbm10, Hnrnpa3; more preferably, the cell trans-differentiation factor is selected from at least one gene, or at least one RNA, or at least one protein encoded by a gene, selected from Amigo1, Fam126a, Gjb2, or Gprc5c.

12. The method according to claim 11, characterized in that said inhibitor is included in a drug, said drug is formulated for administration in vivo to the nervous system, visual system and auditory system, for example, in vivo administration to the striatum, substantia nigra, subthalamic nucleus, spinal cord, hypothalamus, dorsal midbrain, cerebral cortex, hippocampus, cerebellum, subretinal space, vitreous cavity, inner ear cochlea and vestibulum, preferably to the striatum, substantia nigra, subretinal space and vitreous cavity.

13. The method according to claim 11, characterized in that the disease associated with neuronal dysfunction or death is a neurological disease, and the neurological disease is preferably selected from Parkinson's disease, visual system disease associated with RGC or photoreceptor dysfunction or death, stroke, Alzheimer's disease, brain injury, Huntington's disease, epilepsy, depression, sleep disorders, cerebral ischemia, motor neuron disease, amyotrophic lateral sclerosis, spinal muscular atrophy, ataxia, PolyQ disease, schizophrenia, addiction, Pick's disease, blindness, and deafness, more preferably Parkinson's disease and visual system disease associated with RGC or photoreceptor dysfunction or death;
the visual system disease associated with RGC dysfunction or death is preferably selected from visual impairments caused by RGC cell death, glaucoma, age-related RGC degeneration, optic nerve damage, age-related macular degeneration (AMD), diabetic retinopathy, retinal ischemia or hemorrhage, Leber's hereditary optic neuropathy, or combinations thereof; and the visual system disease associated with photoreceptor dysfunction or death is more preferably selected from photoreceptor degeneration or death caused by injury or degenerative diseases, macular degeneration, retinitis pigmentosa, blindness associated with diabetes, night blindness, color blindness, inherited blindness, congenital achromatopsia, or combinations thereof.

14. The method according to claim 11, characterized in that the neuron is a dopamine neuron, a 5-HT neuron, an NE neuron, a ChAT neuron, a GABA neuron, a glutamatergic neuron, a motor neuron, a photoreceptor cell (such as rod cells and cone cells), a retinal ganglion cell (RGC), a cochlear nerve cell (such as cochlear spiral ganglion cells and vestibular neurons), or a medium spiny neuron (MSN), or a combination thereof; preferably, the neuron is the dopamine neuron, the retinal ganglion cell or the photoreceptor cell.

15. The method according to claim 13, characterized in that the inhibitor is brought into contact with non-neuronal cells in vitro, resulting in their conversion into neurons or neuronal precursor cells in vitro; or is administered directly to the object in need to induce the conversion of non-neuronal cells into neurons or neuronal precursor cells in vivo.

16. The method according to claim 11, characterized in that the inhibitor is selected from: a gene editor, an epigenetic regulator, an antibody, a small molecule compound, mRNA, microRNA, siRNA, shRNA, antisense oligonucleotide, binding protein or protein domain, peptide, nucleic acid aptamer, PROTAC, an expression vector containing a promoter, a protein mimetic, or a synthetic or modified form or combination thereof;
preferably, the gene editing tool comprises: (a) a gene editing system or its expression vector, wherein the gene editing system is selected from: CRISPR system (including CRISPR/dCas system), ZFN system, TALEN system, RNA editing system, or a combination thereof; and/or (b) one or more desired gRNA or its expression vector, wherein the gRNA guides the gene editing protein to specifically bind to the DNA or RNA of the target gene. more preferably, the CRISPR system comprises a coding nucleic acid for cas enzyme or its functional domain and a gRNA targeting the cell trans-differentiation factors; more preferably, the cas enzyme is Cas13d, CasRx, Cas13X, Cas13a, Cas13b, Cas13c, or Cas13Y; more preferably, the cas enzyme is CasRx, Cas13X, or Cas13Y; and most preferably, the cas enzyme is CasRx.

17. A pharmaceutical composition or kit or reagent kit comprising the inhibitor according to claim 11, preferably, the pharmaceutical composition or kit or reagent kit further comprises an expression vector; more preferably, the expression vector is a viral vector, a lipid nanoparticle (LNP), a liposome, a cationic polymer (such as PEI), a nanoparticle, an exosome, or a virus-like particle; more preferably, the expression vector is a viral vector or lipid nanoparticle; more preferably, the viral vector is an adeno-associated virus (AAV) vector, a self-complementary adeno-associated virus (scAAV) vector, an adenovirus vector, a lentivirus vector, a retrovirus vector, a herpesvirus vector, an SV40 vector, or a poxvirus vector, or a combination of at least two, and most preferably, the viral vector is an AAV vector.

18. The pharmaceutical composition or kit according to claim 17, wherein the inhibitor comprises:
(a) Gene editing system or its expression vector, the editing system includes: CRISPR system (including CRISPR/dCas system), ZFN system, TALEN system, RNA editing system, or their combinations; and/or
(b) one or more gRNA or their expression vectors, where the gRNA guides the gene editing protein to specifically bind to the DNA or RNA of the target gene;
the CRISPR gene editing system (including CRISPR systems that target both DNA and RNA) is preferred;
more preferably, the drug combination or kit comprise a single type of gRNA, or 2, 3, 4, 5, 6, 7, 8, 9, 10, 11, or 12 different types of gRNA that targets the DNA or mRNA sequence of the target gene, alternatively, the gRNA expression vector encodes the gRNA which is a single type of gRNA or 2, 3, 4, 5, 6, 7, 8, 9, 10, 11, or 12 different types of gRNA that targets the mRNA sequence of the target gene.

19. The pharmaceutical composition or kit according to claim 18, characterized in that the inhibitor is selected from gene editing tools of CRISPR, including nucleic acids encoding cas proteins, promoters of cas proteins, gRNA targeting cell trans-differentiation factors, and promoters of gRNA;
preferably, the gene editing tool of CRISPR includes:
i) a nucleotide sequence encoding the gene editing protein operably linked to the promoter that causes expression of the gene editing protein, wherein the promoter is a broad-spectrum promoter or a specific promoter, wherein the broad-spectrum promoter is selected from CMV, CBH, CAG, PGK, SV40, EF1A, EFS, pGlobin promoters, and the specific promoter is preferably a glial cell-specific promoter or a Müller glial cell (MG) cell-specific promoter, more preferably, the glial cell-specific promoter is selected from GFAP promoter, ALDH1L1 promoter, EAAT1/GLAST promoter, glutamine synthetase promoter, S100β promoter, and EAAT2/GLT-1 promoter, NG2 promoter, CD68 promoter, F4/80 promoter, or the MG cell-specific promoter is selected from GFAP promoter, ALDH1L1 promoter, Glast (also known as Slc1a3) promoter, and Rlbp1 promoter; and
ii) at least one nucleotide sequence encoding gRNA targeting the mRNA or DNA sequence, wherein the nucleotide sequence is operably linked to a promoter that causes expression of the gRNA in mammalian cells, such as the U6 promoter.

20. A pharmaceutical composition or kit according to claim 17, wherein the pharmaceutical composition or kit is locally administered to the body of a subject in need, preferably selected from the following parts of the nervous system: retina, striatum, substantia nigra, inner ear, spinal cord, prefrontal cortex, motor cortex, thalamus, ventral tegmental area (VTA), hippocampus, cerebellum, brainstem, or inner ear cochlea or vestibule; more preferably, the pharmaceutical is administered to the striatum, substantia nigra, retina, and vitreous cavity of an subject in need; or
the pharmaceutical composition or kit induces glial cells to transform into neuronal cells in vitro, and then neuronal cells are given to a subject in need, preferably selected from the following types of glial cells: astrocytes, oligodendrocytes, microglia, NG2 cells, Müller glia cells, glioblastoma cells, or spiral ganglion glia cells, more preferably, the glial cells are selected from astrocytes, Müller glia cells, or spiral ganglion glia cells.

21. A pharmaceutical composition or kit according to claim 17, wherein the composition or the kit further comprises i) one or more dopamine neuron-related factors, or ii) at least one expression vector for expressing one or more dopamine neuron-related factors in the glial cells; preferably, the dopamine neuron-related factors are selected from one or more combinations of Lmx1a, Lmx1b, FoxA2, Nurr1, Pitx3, Gata2, Gata3, FGF8, BMP, En1, En2, PET1, Pax family proteins, SHH, Wnt family proteins, and TGF-β family proteins.

22. A pharmaceutical composition or kit according to claim 17, wherein the composition further comprises i) one or more factors selected from β-catenin, Oct4, Sox2, Klf4, Crx, Brn3a, Brn3b, Math5, Nr2e3, or Nr1, and/or ii) at least one expression vector for expressing one or more factors selected from β-catenin, Oct4, Sox2, Klf4, Crx, Brn3a, Brn3b, Math5, Nr2e3, or Nr1 in the glial cells.

23. A pharmaceutical composition or kit according to claim 17, the inhibitor is prepared to use in cell transfection, cell infection, endocytosis, injection, intracranial administration, spinal cord administration, intraocular administration, intraaural administration, inhalation, extraintestinal administration, intravenous administration, intramuscular administration, subcutaneous administration, surface administration, or oral administration, as well as for ex vivo induction of differentiation, trans-differentiation or reprogramming, and transplantation of differentiated, transdifferentiated or reprogrammed cells back into the body.

24.-27. (canceled)
